(19) United States
(12) Patent Application Publication
(10) Pub. No.: US 2008/0171354 A1
(43) Pub. Date: Jul. 17, 2008

(54) NATRIURETIC PEPTIDE RATIO FOR DIAGNOSING CARDIAC DYSFUNCTIONS

(76) Inventors: Georg Hess, Mainz (DE); Andrea Horsch, Mannheim (DE)

Correspondence Address: ROCHE DIAGNOSTICS OPERATIONS INC., 9115 Hague Road, Indianapolis, IN, 46250-0457, US

(21) Appl. No.: 11/838,925

(22) Filed: Aug. 15, 2007

Related U.S. Application Data
(63) Continuation of application No. PCT/EP2006/060059, filed on Feb. 17, 2006.

(30) Foreign Application Priority Data
Feb. 17, 2005 (EP) EP 05003477.6

(51) Int. Cl. C12Q 1/02

(52) U.S. Cl. 435/29

(57) ABSTRACT

The present invention is a method for diagnosing a cardiac dysfunction in a subject comprising the steps of measuring, preferably in vitro, the level of a BNP-type peptide in a sample from the subject, measuring, preferably in vitro, the level of an ANP-type peptide in a sample from the subject, calculating the ratio of the measured level of the ANP-type peptide to the measured level of the BNP-type peptide comparing the calculated ratio to at least one known ratio indicative of the presence or absence of a cardiac dysfunction. Preferred markers according to the present invention are ANP, NT-proANP, BNP, NT-proBNP, which belong to the class of natriuretic peptides. Particularly, the present invention relates to diagnosing a diastolic dysfunction and/or (distinguishing a diastolic from a systolic dysfunction. Furthermore, the present invention relates to diagnostic kits (comprising an ANP-type and a BNP type peptide) as well as methods of treatment and methods for deciding about treatment.

### RELATED APPLICATIONS

This application is a continuation of PCT/EP2006/0600059 filed Feb. 17, 2006 and claims priority to EP 05003477.6 filed Feb. 17, 7005.

### FIELD OF THE INVENTION

The present invention relates to the use of natriuretic peptides for diagnosing cardiac dysfunctions, particularly diastolic dysfunctions.

### BACKGROUND OF THE INVENTION

An aim of modern medicine is to provide personalized or individualized treatment regimens. Those are treatment regimens which take into account a patient's individual needs or risks. Of particular importance are cardiac dysfunctions and heart failure.

Cardiac dysfunctions and heart failure belong to the most common causes of morbidity and mortality in the northern hemisphere. Cardiac dysfunctions can be divided into systolic and diastolic dysfunctions. Diastolic and systolic dysfunctions relate to the filling phases of the heart which are predominantly affected.

The human heart comprises four chambers: Two thin-walled atria and two muscular ventricles. The blood flows into the right atrium, is pumped into the right ventricle and from there into the lungs. The blood is oxygenated in the lungs and flows into the left atrium, from where it is pumped into the left ventricle. The left ventricle pumps the blood into the body. The atria can be understood to serve as “reservoirs”, whereas, the major pump functions are carried out by the ventricles. However, the atria pump blood actively into the ventricles and thus contribute about 10% to the total pump function of the heart.

Systolic dysfunctions affect the phase of ejecting the blood from the left ventricle into the circulation. Thus, systolic dysfunctions are commonly characterized by a reduced amount of blood ejected from the left ventricle. Systolic dysfunctions are usually symptomatic as the body is not adequately supplied with oxygenated blood, particularly under conditions of physical activity. The patients may complain of fatigue and exhaustion.

In contrast, diastolic dysfunctions affect the phase of between the ejection phases of the left ventricle. Diastolic dysfunctions can remain asymptomatic for much longer. Causes for diastolic dysfunction are abnormal relaxation, filling, or dispensability of the left ventricle.

Both systolic and diastolic dysfunctions may eventually lead to heart failure. Although the mortality rate among patients with diastolic heart failure is lower than the mortality rate of patients with systolic heart failure, it is important to note that due to a large lack of obvious symptoms diastolic dysfunctions may remain undetected for much longer than systolic dysfunctions. Therefore, improved diagnosis is important.

Early detection of diastolic dysfunction would allow early therapeutic intervention and might help to prevent overt heart failure. It would also allow to devise treatment methods specifically tailored to diastolic dysfunction. However, diagnosis of diastolic dysfunction is difficult. Physical examination, electrocardiogram, and chest radiograph do not provide information that distinguishes diastolic from systolic heart failure (Aurigemma, G. P., and Gaasch, W. H. (2004). Diastolic Heart Failure. The New England Journal of Medicine, vol. 35(11), pp. 1097-1105). Therefore, the currently most important diagnostic tool in this context is echocardiography. However, echocardiography requires an expensive technical equipment and a certain degree of experience on the part of the clinician. Thus, echocardiography is not used for regular screening, of patients but only in the case of a suspected cardiac dysfunction. Importantly, a more severe or more advanced diastolic dysfunction may appear in the echocardiogram with a “pseudo normal” pattern and thus may remain undetected.

The use of biochemical or molecular markers for diagnostic purposes is known as such. However, currently it is not known which marker(s) yield valuable information for diagnosis of diastolic dysfunction.

Lubien et al. have reported that brain natriuretic peptide (BNP) may be useful in diagnosis of diastolic dysfunction (Lubien, E., DeMaria, A., Krishnaswamy, P., et al. (2002). Utility of B-Natriuretic Peptide in Detecting Diastolic Dysfunction. Comparison with Doppler Velocity Recordings. Circulation, vol. 105, pp. 595-601). However, Ambrosi et al. have voiced considerable doubts concerning the validity of these results (Ambrosi, P., Oddoze, C., Habib, G. et al. (2002). Utility of B-Natriuretic Peptide in Detecting Diastolic Dysfunction. Comparison with Doppler Velocity Recordings. Letter to the Editor. Circulation, vol. 106, p. e70).

It has been mentioned that brain natriuretic peptide levels are not as high in diastolic heart failure than in systolic heart failure, “but more data are needed to assess the role of brain natriuretic, peptide in the diagnosis of diastolic heart failure” (Aurigemma, G. P., and Gaasch, W. H. (29004). Diastolic Heart Failure. The New England Journal of Medicine, vol. 351 (I), pp. 1097-1105).

Wang et al. measured both atrial natriuretic peptide (ANP) and BNP in patients included in the Framingham Heart Study (Wang, T. J., Larson, M. G., Levy, D., Benjamin, E. J. et al. (2004) Plasma Natriuretic Peptide Levels and the Risk of Cardiovascular Events and Death. The New England Journal of Medicine, vol. 350(7), pp. 655-663). They conclude that their data raises the possibility that measurement of natriuretic peptides may aid the early detection of cardiovascular disease, but that additional investigations were needed.

Thus, in the state of the art there appears to be no biochemical marker which could be used to diagnose a diastolic dysfunction. Furthermore, no biochemical marker is known which allows distinguishing a diastolic from a systolic dysfunction.

### SUMMARY OF THE INVENTION

Therefore, it is an object of the present invention to provide methods and means to diagnose a cardiac dysfunction. Furthermore, it is an object of the present invention to provide methods and means to diagnose a diastolic dysfunction, in particular to provide methods and means to distinguish a diastolic from a systolic dysfunction.

In a first embodiment, the object is achieved by a method for diagnosing a cardiac dysfunction in a subject, comprising the steps of
measuring, preferably in vitro, the level of a BNP-type peptide in a sample from the subject,
measuring, preferably in vitro, the level of an ANP-type peptide in a sample from the subject,
calculating the ratio of the measured level of the ANP-type peptide to the measured level of the BNP-type peptide,
comparing the calculated ratio to at least one known ratio indicative of the presence or absence of a cardiac dysfunction,

In an optional step, the cardiac dysfunction in the subject is diagnosed. The method may also comprise the step of taking a body fluid or tissue sample from the patient. Within the present invention, the taking of the body fluid or tissue sample can preferably be carried out by non-medical staff (i.e. not having an education necessary for carrying out the profession of a physician). This applies in particular if the body sample is blood.

In the context of the present invention, it has been found that the ratio of the level of an ANP-type peptide to the level of an BNP-type peptide can be used to diagnose a cardiac dysfunction. In particular, it has been found that the ratio allows diagnosing a diastolic dysfunction. Furthermore, it has been found that the ratio allows distinguishing a diastolic dysfunction from a systolic dysfunction.

Unexpectedly, it has been found that the combined evaluation of ANP-type and BNP-type peptide levels, e.g. expressed as their ratio to each other, leads to improved diagnostic information. Therefore, in a preferred embodiment of the invention, the diagnostic information of the levels of ANP-type and BNP-type peptides is combined.

Combining the information of the levels of the ANP-type and BNP-type peptides may serve to normalize the diagnostic information from each marker in relation to the other in the individual patient. For example, the BNP-type peptide level of an individual patient may be high in response to volume overload, arterial hypertension, or general strain on the heart. However, these factors will in most cases also affect the level of the ANP-type peptides, which will also be increased. Therefore, the combined information, e.g. expressed as the ratio, allows an improved diagnosis, as the diagnostic information is derived from a change in the relation of the levels of ANP-type peptide to BNP-type peptide and not from the absolute level of one of these markers.

### BRIEF DESCRIPTION OF THE DRAWINGS

FIG. 1 shows the results of measurements of NT-proANP and NT-proBNP in patients from the sequential study described in Example 3. DG, diagnostic group (as described in Example 3); N, number of subjects; MIN, minimal value observed; MAX, maximal value observed, MEAN, mean value observed; MEDIAN, median of the values observed; LVEF left ventricular ejection fraction (in number N of patients).

FIG. 2 shows the results of measurements of NT-proANP and NT-proBNP in patients from the sequential study described in Example 3. DG, diagnostic group (as described in Example 3); N, number of subjects; MIN, minimal value observed; MAX, maximal value observed, MEAN, mean value observed; MEDIAN, median of the values observed; LVEF left ventricular ejection fraction (in number N of patients). Values in rows designated “percentile” indicate the levels measured in each percentile indicated.

FIG. 3 shows the results of measurements of NT-proANP and NT-proBNP in patients from the sequential study described in Example 3. DG, diagnostic group (as described in Example 3): N, number of subjects; MIN, minimal value observed; MAX, maximal value observed, MEAN, mean value observed; MEDIAN, median of the values observed; LVEF left ventricular ejection fraction (in number N of patients); ED, electrocardiographic diagnoses as described in Example 3. FIG. 3 also shows the levels measured for different groups according to the LVEF measured in those subjects.

### DETAILED DESCRIPTION OF THE INVENTION

The invention takes advantage of certain biochemical or molecular markers. The terms “biochemical marker” and “molecular marker” are known to the person skilled in the art. In particular, biochemical or molecular markers are gene expression products which are differentially expressed (i.e. upregulated or downregulated) in presence or absence of a certain condition, disease, or complication. Usually, a molecular marker is defined as a nucleic acid (such as an mRNA), whereas a biochemical marker is a protein or peptide. The level of a suitable biochemical or molecular marker can indicate the presence or absence of the condition, disease, risk, or complication, and thus allow diagnosis.

The present invention particularly takes advantage of ANP-type and BNP-type peptides as biochemical or molecular markers. ANP-type and BNP-type peptides belong to the group of natriuretic peptides (see e.g. Bonow, R. O. (1996). New insights into the cardiac natriuretic peptides. Circulation 93: 1946-1950). ANP-type peptides comprise pre-proANP, proANP, NT-proANP, and ANP. BNP-type peptides comprise pre-proBNP, proBNP, NT-proBNP, and BNP.

The pre-pro peptide (134 amino acids in the case of pre-proBNP) comprises a short signal peptide, which is enzymatically cleaved off to release the pro peptide (108 amino acids in the case of proBNP). The pro peptide is further cleaved into an N-terminal pro peptide (NT-pro peptide, 76 amino acids in case of NT-proBNP) and the active hormone (32 amino acids in the case of BNP, 28 amino acids in the case of ANP).

Preferred natriuretic peptides according to the present invention are NT-proANP, ANP, NT-proBNP, BNP, and variants thereof. ANP and BNP are the active hormones and have a shorter half-life than their respective inactive counterparts, NT-pro ANP and NT-proBNP BNP is metabolised in the blood, whereas NT-proBNP circulates in the blood as an intact molecule and as such is eliminated renally. The in-vivo half-life of NT-proBNP is 120 min. longer than that of BNP, which is 20 min (Smith M W, Espiner E A, Yandle T G, Charles C J, Richards A M. Delayed metabolism of human brain natriuretic peptide reflects resistance to neutral endopeptidase J Endocrinol. 2000; 167: 239-46.).

BNP is produced predominantly (albeit not exclusively) in the ventricle and is released upon increase of wall tension. Thus, an increase of released BNP reflects predominantly dysfunctions of the ventricle or dysfunctions which originate in the atria but affect the ventricle, e.g. through impaired inflow or blood volume overload.

In contrast, ANP is produced and released exclusively from the atrium. The level of ANP may therefore predominantly reflect atrial function.

Preanalytics are robust with NT-proBNP, which allows easy transportation of the sample to a central laboratory (Mueller T, Gegenhuber A, Dieplinger B, Poelz W, Haltmayer M. Long-term stability of endogenous B-type natriuretic peptide (BNP) and amino terminal proBNP (NT-proBNP) in frozen plasma samples. Clin Chem Lab Med 2004; 42: 942-4.). Blood samples can be stored at room temperature for several days or may be mailed or shipped without recovery loss. In contrast, storage of BNP for 48 hours at room temperature or at 4° Celsius leads to a concentration loss of at least 20% (Mueller T, Gegenhuber A, et al., Clin Chem Lab Med 2004; 42: 942-4, supra; Wu A H, Packer M, Smith A, Bijou R, Fink D, Mair J, Wallentin L, Johnston N, Feldcamp C S, Haverstick D M, Ahnadi C E, Grant A, Despres. N, Bluestein B, Ghani F. Analytical and clinical evaluation of the Bayer ADVIA Centaur automated B-type natriuretic peptide assay in patients with heart failure: a multisite study. Clin Chem 2004; 50: 867-73.).

Therefore, depending on the time-course or properties of interest, either measurement of the active or the inactive, forms of the natriuretic peptide can be advantageous.

The term “variants” in this context relates to peptides substantially similar to said peptides. The term “substantially similar” is well understood by the person skilled in the art. In particular, a variant may be an isoform or allele which shows amino acid exchanges compared to the amino acid sequence of the most prevalent peptide isoform in the human population. Preferably, such a substantially similar peptide has a sequence similarity to the most prevalent isoform of the peptide of at least 80%, preferably at least 85%, more preferably at least 90%, most preferably at least 95%. Substantially similar are also proteolytic degradation products which are still recognized by the diagnostic means or by ligands directed against the respective full-length peptide.

The term “variant” also relates to a post-translationally modified peptide such as glycosylated peptide. A “variant” is also a peptide which has been modified after collection of the sample, for example by covalent or non-covalent attachment of a label, particularly a radioactive or fluorescent label, to the peptide. Measuring the level of a peptide modified after collection of the sample is understood as measuring the level of the originally non-modified peptide.

Diagnosing according to the present invention includes determining, monitoring, confirmation, subclassification and prediction of the relevant dysfunction or disease. Determining relates to becoming aware of the dysfunction or disease. Monitoring relates to keeping track of an already diagnosed dysfunction or disease, e.g. to analyze the progression of the dysfunction or disease or the influence of a particular treatment on the progression of dysfunction or disease Confirmation relates to the strengthening or substantiating a diagnosis already performed using other indicators or markers. Subclassification relates to further defining a diagnosis according to different subclasses of the diagnosed dysfunction or disease, e.g. defining according to mild and severe forms of the dysfunction or disease. Prediction relates to prognosing a dysfunction or disease before other symptoms or markers have become evident or have become significantly altered.

Preferably, the diagnostic information gained by the means and methods according to the present invention is interpreted by a trained physician. Preferably, any decision about further treatment in an individual subject is also made by a trained physician. If deemed appropriate, the physician will also decide about further diagnostic measures.

The term “subject” according to the present invention relates to a healthy individual, an apparently healthy individual, or a patient. The subject may have no known history of cardiovascular disease, and/for no or little symptoms of a cardiac risk or complication, and/or he is not being treated for a cardiac disease, risk, or complication.

A “patient” is an individual suffering from a disease. Particularly, the patient may be suffering from cardiac disease or be suspected of having a diastolic or systolic dysfunction.

The present invention broadly concerns the diagnosis of cardiac dysfunctions. Patients suffering from a cardiac dysfunction may be individuals suffering from stable angina pectoris (SAP) and individuals with acute coronary syndromes (ACS). ACS patients can show unstable angina pectoris (UAP) or these individuals have already suffered from a myocardial infarction (MI). MI can, be an ST-elevated MI or a non-ST-elevated MI. The occurring of an MI can be followed by a left ventricular dysfunction (LVD). Finally, LVD patients undergo congestive heart failure (CHF) with a mortality rate of roughly 15%.

Cardiac dysfunctions according to the present invention also include coronary heart disease, heart valves defects (e.g. mitral valve defects) dilative cardiomyopathy, hypertrophic cardiomyopathy, and heart rhythm defects (arrhythmias).

The cardiac dysfunctions according to the present invention may be “symptomatic” or “asymptomatic”. Symptoms of cardiac dysfunctions can be classified into a functional classification system established for cardiovascular diseases according to the New York Heart Association (NYHA). Patients of Class I have no obvious symptoms of cardiovascular disease. Physical activity not limited, and ordinary physical activity does not cause undo fatigue, palpitation, or dyspnea (shortness of breath). Patients of class II have slight limitation of physical activity. They are comfortable at rest, but ordinary physical activity results in fatigue, palpitation, or dyspnea. Patients of class III show a marked limitation of physical activity. They are comfortable at rest, but less than ordinary activity causes fatigue, palpitation, or dyspnea. Patients of class IV are unable to carry out any physical activity without discomfort. They show symptoms of cardiac insufficiency at rest. If any physical activity is undertaken, discomfort is increased.

Another indicator of cardiac dysfunction, particularly systolic dysfunction, is the “left ventricular ejection fraction” (LVEF) which is also known as “ejection fraction”. People with a healthy heart usually have an unimpaired LVEF, which is generally described as above 50%. Most people with a systolic dysfunction which is symptomatic generally have an LVEF of 40% or less.

Particularly, the present invention relates to the diagnosis of diastolic dysfunction. More particularly, the present invention relates to distinguishing a diastolic from a systolic dysfunction. The term “diastolic dysfunction” is known to the person skilled in the art. In diastolic dysfunction, the ejection fraction is normal and the end-diastolic pressure is elevated; there is diminished capacity to fill at low left-atrial pressures. In contrast, in “systolic dysfunction” the LVEF is reduced and the end-diastolic pressure is normal. Diastolic dysfunction may be assessed by continuously measuring the flow velocity across the mitral valve (i.e. from left atrium to left ventricle) using Doppler echocardiography. Normally the velocity of inflow is more rapid in early diastole than during atrial systole (atrial systole refers to the contraction of the atrium with blood flow into the ventricle); with impaired relaxation the rate of early filling declines, whereas the rate of presystolic filling increases. With more severe impairment of filling the pattern becomes (“pseudonormal” and early ventricular filling becomes more rapid as left atrial pressure upstream of the still left ventricle rises.

Diastolic function is influenced by the passive elastic properties of the left ventricle and by the process of active relaxation. Abnormal passive elastic properties generally are caused by a combination of increased myocardial mass and alterations in the extramyocardial collagen network. The effects of impaired active myocardial relaxation can further stiffen the ventricle. As a result, left ventricular diastolic pressure in relation to volume is increased, chamber compliance (contractibility of the ventricle) is reduced, the time-course of filling is altered, and the diastolic pressure is elevated. Thus, mechanisms for diastolic dysfunction include abnormal relaxation, filling, or distensibility (i.e. increased chamber stiffness), and chamber dilation of the left ventricle. A further mechanism is pericardial restraint. Further mechanisms of diastolic dysfunction, particularly in hypertrophic or ischemic heart disease include fibrosis, cellular disarray (both of which increase chamber stiffness), hypertrophy (which increases chamber stiffness but also decreases relaxation of the ventricle), asynchrony, abnormal loading, ischemia, and abnormal calcium flux (the latter four mechanisms decrease relaxation of the ventricle).

Advantageously, the present invention allows distinguishing a diastolic dysfunction from a systolic dysfunction. The term “systolic dysfunction” is known to the person skilled, in the art and has already been explained above.

In this context, it should be noted that certain patients may show a mixed form of diastolic and systolic dysfunction. For example, a severe diastolic dysfunction may lead to a systolic dysfunction and the character of the dysfunction under this borderline condition may be mixed. It is evident to the person skilled in the art, that such a mixed form of diastolic and systolic dysfunction will most likely be present at the border values between the ratios (of ANP-type to BNP-type peptide) indicative of diastolic and systolic dysfunction, e.g. in a range of 3.5 to 7 (pg/ml of NT-proANP to pg/ml of NT-proBNP). Thus, the present invention may also be understood as being able to distinguish a primarily diastolic from a primarily systolic dysfunction.

In another preferred embodiment, the present invention also relates to a method for diagnosing the severity of a diastolic dysfunction. It has been found that the ratio of ANP-type to BNP-type peptide is “inversely correlated” with the severity of the diastolic dysfunction. This means that the lower the ratio, the more severe is the diastolic dysfunction and vice versa. However, as evident from the context of the specification, a very low ratio (e.g. below 4.5 pg/ml of NT-proANP to pg/ml of NT-proBNP) indicates that the dysfunction is systolic or primarily systolic and a very high ratio indicates that no cardiac dysfunction is present.

Typically, a “less severe” diastolic dysfunction (or “early phase” of a diastolic dysfunction) is brought on by abnormally slow left ventricular relaxation and/or a reduced velocity of early filling.

Typically, a “more severe” diastolic dysfunction (or “advanced phase” of diastolic dysfunction), is mainly characterized by additional abnormalities in chamber compliance.

In Doppler echocardiography, less severe and more severe diastolic dysfunction may be distinguished according to the ratio of the “E-wave” to the “A-wave”: The mild diastolic dysfunction (abnormal relaxation pattern) is brought on by abnormally slow left ventricular relaxation, a reduced velocity of early filling (E-wave), an increase in the velocity associated with atrial contraction (A-wave), and a ratio of F to A that is lower than normal. In the more severe diastolic dysfunction, i.e. the “advanced phase” when left atrial pressure has risen, the E-wave velocity and the E to A ratio is similar to that in normal subjects and results in a “pseudo normal” velocity pattern. Furthermore, in more severe diastolic dysfunction, abnormalities in left ventricular compliance may supervene, resulting in a high E-wave velocity. In these latter two cases, the E-wave of normal to high velocity is a result of high left atrial pressure and a high pressure gradient across the mitral valve in early diastole. The Doppler echocardiogram in diastolic dysfunction is further discussed in Aurigemma and Gaasch, 2004, cited above).

A diastolic dysfunction may result in “diastolic heart failure”. A criterion for “diastolic heart failure” is the presence of a normal LVEF (above 50%) within three days after an episode of heart failure. Preferably objective evidence of diastolic dysfunction is also present (see above, e.g. abnormal left ventricular relaxation, filling or distensibility). Diagnosis of diastolic heart failure may also be made clinically, if there is reliable evidence of congestive heart failure and a normal LVEF, and that objective evidence of diastolic dysfunction obtained in the catheterization laboratory merely confirms diagnosis. This conclusion is consonant with the American College of Cardiology and the American Heart Association guidelines.

The principal difference between systolic and diastolic heart failure is the inability to relax or fill normally (diastolic heart failure) and the inability of the ventricle to contract normally and expel sufficient blood (systolic heart failure). Impaired relaxation or filling of the ventricle leads to an elevation of ventricular diastolic pressure at any given diastolic volume. Failure of relaxation can be functional and transient, as during ischemia, or it can be chronic, e.g. due to a stiffened, thickened ventricle.

According to the present invention, the term “diastolic heart failure” does not encompass conditions such as acute severe mitral regurgitation and other circulatory congestive states (e.g. congestive heart failure), which may also result in heart failure with normal ejection fraction. In these cases one would typically expect a relatively low ratio of ANP-type peptide to BNP-type peptide, e.g. a ratio of less than 5 pg/ml of NT-proANP to pg/ml of NT-proBNP.

In another preferred embodiment, the present invention relates to distinguishing cardiac dysfunctions in which one or both atria are affected from cardiac dysfunctions in which the one or both ventricles are affected. Again, the present invention may also relate to distinguishing the primary character of such dysfunctions, i.e. distinction of an atrial from a ventricular dysfunction. A higher ratio of ANP-type peptide to BNP-type peptide will indicate that the atrium is affected, whereas a lower ratio will indicate that the ventricle is affected. In more general terms, the invention allows to distinguish whether the dysfunction is primarily atrial or primarily ventricular.

Primary malfunctions of the atrium, e.g. atrial fibrillation, may result in a failure of contraction of the atrium with the consequence that the blood does not actively reach the ventricle. Atrial fibrillation causes incoordinate contractions of the atrial musculature so that a contraction does not take place anymore. Similarly, malfunctions in the ventricle may impede the blood flow from the atrium into the ventricle.

Furthermore, in advanced cardiac dysfunctions, e.g. in the case of valve dysfunctions, a backflow from the ventricle into the atrium is possible, e.g. caused by incomplete valve closure. Similar phenomena may be observed e.g. after myocardial infarction affecting the muscles which move the valves (papillary muscles). This results in increased strain on the atrium by backflow from the ventricle to the atrium (regurgitation).

For example, the subject is analyzed for atrial fibrillation (e.g. by electrocardiography). It should be noted that atrial fibrillation may cause an increase in the levels of the ANP-type and/or BNP-type peptide and a decrease in the ratio of the ANP-type peptide to the BNP-type peptide (see also Example 3). In subjects suffering from atrial fibrillation, the diagnostic information gained by the ratio is preferably interpreted with care and is preferably confirmed by other means described in this specification, e.g. by echocardiography. Furthermore, the calculated ratio may be corrected (i.e. increased) to achieve better diagnosis in such subjects.

In the context of the present invention, it has been found that even measuring the BNP-type peptide alone may be sufficient for diagnosing a cardiac dysfunction, particularly for diagnosing a diastolic dysfunction or for distinguishing a diastolic dysfunction from a systolic dysfunction. Therefore, in another embodiment, the present invention relates to a method for diagnosing a cardiac dysfunction in a subject, comprising the steps of measuring, preferably in vitro, the level of a BNP-type peptide in a sample from the subject, and comparing the level of the BNP-type peptide to at least one known level indicative of the presence or absence of a cardiac dysfunction. The method may include an optional step of diagnosing the cardiac dysfunction in the subject. This embodiment particularly relates to diagnosing a diastolic dysfunction. All other embodiments of the present invention may be adapted analogously to measuring the BNP-type peptide alone.

In general, the higher the level of the BNP-type peptide, the higher is the likelihood of the presence of a diastolic dysfunction and/or the more severe is the diastolic dysfunction. However, a cry high level of the BNP-type peptide (e.g. above 700 pg/ml, preferably above 1000 pg/ml of NT-proBNP) indicates that the dysfunction is systolic or primarily systolic.

The method according to the present invention comprises the step of diagnosing the dysfunction by comparing the calculated ratio to at least one known ratio indicative of the presence of a cardiac dysfunction, particularly of a diastolic or systolic dysfunction.

It is evident that the combined information from ANP-type and BNP-type peptide ratio may also be expressed differently, e.g. as the ratio of the level of the BNP-type-peptide to the ANP-type peptide. Any concentrations (molar or by weight) can be calculated easily. These forms of measurement represent the same invention and are considered to be within the scope of the term “ratio of the ANP-type to the BNP-type peptide”.

The person skilled in the art is able to determine known level(s) or ratio(s), see also Example 2. For example, the median of the measured levels or ratios in a population of subjects suffering from a particular dysfunction can be used. Analogously, a population of control subjects may be investigated. Evaluating the levels in further subjects, e.g. in cohort studies, can help to refine the known levels or ratios.

The terms “control” or “control sample” are easily understood by the person skilled in the art. Preferably, the “control” relates to an experiment or test carried out to provide a standard, against which experimental results can be evaluated. In the present context, the standard preferably relates to the level of the peptide of polypeptide of interest associated with a particular disease status. Thus, a “control” is preferably a sample taken to provide such a standard. E.g., the control sample may be derived from one or more healthy subjects, or from one or more patients representative of a particular-disease status. The control sample may also have been derived from the subject at an earlier time.

The known level may also be a “reference value”. The person skilled in the art is familiar with the concept of reference values (or “normal values”) for biochemical or molecular markers. In particular, the term reference value may relate to the actual value of the level in one or more control samples or it may relate to a value derived from the actual level in one or more control samples. Preferably, samples of at least 3, more preferably at least 15, more preferably at least 50, more preferably at least 100, most preferably at least 400 subjects are analyzed to determine the reference value.

In the most simple case, the reference value is the same as the level measured in the control sample or the average of the levels measured in a multitude of control samples. However, the reference value may also be calculated from more than one control sample. E.g., the reference value may be the arithmetic average of the level in control samples representing the control status (e.g. healthy, particular condition, or particular disease state). Preferably, the reference value relates to a range of values that can be found in a plurality of comparable control samples (control samples representing the same or similar disease status), e.g. the average ±one or more times the standard deviation. Similarly, the reference value may also be calculated by other statistical parameters or methods, for example as a defined percentile of the level found in a plurality of control samples, e.g. a 90%, 95%, or 99% percentile. The choice of a particular reference value may be determined according to the desired sensitivity, specificity or statistical significance (in general, the higher the sensitivity, the lower the specificity and vice versa). Calculation may be carried out according, to statistical methods known and deemed appropriate by the person skilled in the art.

Examples for known levels or ratios are given below. It will be possible to further refine such levels or ratios. The particular known levels or ratios given in this specification may serve as a guideline to diagnose the cardiac dysfunction. As known and well-accepted in the aft, actual diagnosis in the individual subject is preferably carried out through individual analysis by a physician, e.g. depending on weight, age, general health status and anamnesis of the individual subject.

For example, a ratio of the plasma levels of less than 20, preferably of less than 17, (pg/ml of NT-proANP to pg/ml of NT-proBNP) indicates the presence of a cardiac dysfunction. In another example, a ratio of the plasma levels of more than 20, preferably more than 23, (pg/ml of NT-proANP to pg/ml of NT-proBNP) indicates the absence of a cardiac dysfunction.

Furthermore, a ratio of the plasma levels in the range of 6 to 20, preferably of 7 to; 17, (pg/ml of NT-proANP to pg/ml of NT-proBNP) indicates the presence of a diastolic dysfunction. A ratio in the range of 15 to 20 (pg/ml of NT-proANP to pg/ml of NT-pro BNP) indicates the presence of a less severe diastolic dysfunction. A ratio in the range of 6 to 15 (pg/ml of NT-proANP to pg/ml of NT-proBNP) indicates the presence of a more severe diastolic dysfunction. A ratio of less than 6, preferably less than 4.5, indicates the presence of a systolic dysfunction.

For example, a plasma level in the range of 125 to 700 pg/ml of NT-proBNP may indicate the presence of a diastolic dysfunction. A plasma level in the range of 125 to 250 pg/ml of NT-proBNP may indicate the presence of a less severe diastolic dysfunction. A plasma level in the range of 250 to 700 pg/ml of NT-proBNP may indicate the presence of a more severe diastolic dysfunction. A plasma level of more than 700 pg/ml, preferably of more than 1000 pg/ml of NT-proBNP may indicate the presence of a primarily systolic dysfunction. At a level of less than 125 pg/ml, preferably of less than 80 pg/ml, the presence of a diastolic dysfunction is unlikely.

The values for levels and/or ratios be expressed in different manner, the values may be expressed in molar units instead of the weight per volume and vice versa. Similarly, a ratio of BNP-type peptide to ANP-type may be used instead of the ratio of ANP-type peptide to BNP-type peptide and the values may be recalculated accordingly.

In another preferred embodiment, additional diagnostic parameters of cardiac disease are measured, particularly chosen from the group consisting of (a) left ventricular ejection fraction (LVEF), (b) echocardiogram (c) anamnesis (medical history), in particular concerning angina pectoris, (d) electrocardiogram, (e) atrial fibrillation, (f) parameters of thyroid or kidney function, (g) blood pressure, in particular arterial hypertension, (h) thallium scintigram, (i) angiography, (j) catheterization. These additional diagnostic parameters may be determined before or after measuring the BNP-type (and possibly ANP-type) peptide. They may either establish a suspicion of the presence of a cardiac dysfunction or they may serve to further evaluate the diagnostic relevance of a particular level or ratio measured.

In particular, the possibility that a cardiac dysfunction is present may be determined or confirmed by Doppler echocardiography. Doppler echocardiography may also he particularly advantageous to determine or confirm the possibility that a diastolic dysfunction is present. Analysis of the ratio of E-wave to A-wave (Aurigemma and Gaasch, cited above) in the Doppler echocardiogram allows to confirm a diastolic dysfunction.

The diagnostic information from ANP-type and BNP-type peptide as well as their ratio can yield additional or complementary information to the information from Doppler echocardiography. Among individual subjects, the measured level(s) or ratio may deviate considerably, yielding a more differentiated diagnostic information about the function of the atrium or the ventricle. This information may exceed the information gathered by echocardiography.

An impaired LVEF, particularly an LVEF of less than 40%, will indicate that the dysfunction is systolic or primarily systolic and may be used to confirm diagnosis according to other methods or uses provided by the present invention.

The level of a biochemical or molecular marker can be determined by measuring the concentration of the protein (peptide or polypeptide) or the corresponding the transcript. In this context, the term “measuring” relates preferably to a quantitative or semi-quantitative determination of the level.

The level can be measured by measuring the amount or the concentration of the peptide or polypeptide. Preferably, the level is determined as the concentration in given sample. For the purpose of the invention, it may not be necessary to measure the absolute level. It may be sufficient to measure the relative level compared to the level in an appropriate control. Measurement can also be carried out by measuring derivatives or fragments specific of peptide or polypeptide of interest, such as specific fragments contained in nucleic acid or protein digests.

Measurement of nucleic acids, particularly mRNA, can be performed according to any method known and considered appropriate by the person skilled in the art.

Examples for measurement of RNA include Northern hybridization, RNAse protection assays, in situ hybridization, and aptamers, e.g. Sephadex-binding RNA ligands (Srisawat, C., Goldstein I. J., and Engelke, D. R. (2001). Sephadex-binding RNA ligands rapid affinity purification of RNA from complex RNA mixtures. Nucleic Acids Research, vol. 29, no. 2 e4).

Furthermore, RNA can be reversely transcribed to cDNA. Therefore methods for measurement of DNA can be employed for measurement of RNA as well, e.g. Southern hybridization, polymerase chain reaction (PCR), Ligase chain reaction (LCR) (see e.g. Cao, W. (2004) Recent developments in ligase-mediated amplification and detection. Trends in Biotechnology, vol. 22 (1), p. 38-44), RT-PCR, real time RT-PCR, quantitative RT-PCR, and microarray hybridization (see e.g. Frey, B., Brehm, U., and Kübler, G., et al (20002). Gene expression arrays: highly sensitive detection of expression patterns with improved tools for target amplification. Biochemica, vol. 2, p. 27-29).

Measurement of DNA and RNA may also be performed in solution, e.g. using molecular beacons, peptide nucleic acids (PNA), or locked nucleic acids (LNA) (see e.g. Demidov, V. V. (2003). PNA and LNA throw light on DNA. Trends in Biotechnology, vol. 2(1), p. 4-6).

Measurement of proteins or protein fragments can be carried out according to any method known for measurement of peptides or polypeptides of interest. The person skilled in the art is able to choose an appropriate method.

The person skilled in the art is familiar with different methods of measuring the level of a peptide or polypeptide. The term “level” relates to amount or concentration of a peptide or polypeptide in the sample.

Measuring can be done directly or indirectly. Indirect measuring includes measuring of cellular responses, bound ligands, labels, or enzymatic reaction products.

Measuring can be done according to any method known in the art, such as cellular assays, enzymatic assays, or assays based on binding of ligands. Typical methods are described in the following.

In one embodiment, the method for measuring the level of a peptide or polypeptide of interest comprises the steps of contacting the peptide or polypeptide with a suitable substrate for an adequate period of time, measuring the amount of product.

In another embodiment, the method for measuring the level of a peptide or polypeptide of interest comprises the steps of contacting the peptide or polypeptide with a specifically binding ligand, (optionally) removing non-bound ligand, and measuring the amount of bound ligand.

In another embodiment, the method for measuring the level of a peptide or polypeptide of interest comprises the steps of (optionally) fragmenting the peptides or polypeptides of a sample, (optionally) separating the peptides or polypeptides or fragments thereof according to one or more biochemical or biophysical properties (e.g. according to binding to a solid surface or their run-time in a chromatographic setup), determining the amount of one or more of the peptides, polypeptides, or fragments, determining the identity of one or more of the peptides, polypeptides or fragments by mass spectrometry. An overview of mass spectrometric methods is given e.g. by Richard D. Smith (2002). Trends in mass spectrometry instrumentation for proteomics. Trends in Biotechnology, Vol. 20, No. 12 (Suppl.), pp. S3-S7).

Other typical methods for measurement include measuring the amount of a liquid binding specifically to the peptide or polypeptide of interest. Binding according to the present invention includes both covalent and non-covalent binding.

A ligand according to the present invention can be any peptide, polypeptide, nucleic acid, or other substance binding to the peptide or polypeptide of interest. It is well known that peptides or polypeptides, if obtained or purified from the human or animal body, can be modified, e.g. by glycosylation. A suitable ligand according to the present invention may bind the peptide or polypeptide also via such sites.

Preferably, the ligand should bind specifically to the peptide or polypeptide to be measured. “Specific binding” according to the present invention means that the ligand should not bind substantially to (“cross-react” with) another peptide, polypeptide or substance present in the sample investigated. Preferably, the specifically bound protein or isoform should be bound with at least 3 times higher, more preferably at least 10 times higher and even more preferably at least 50 times higher affinity than any other relevant peptide or polypeptide.

Non-specific binding may be tolerable, particularly if the investigated peptide or polypeptide can still be distinguished and measured unequivocally, e.g. by separation according to its size (e.g. by electrophoresis), or by its relatively higher abundance in the sample.

Binding of the ligand can be measured by any method known in the art. Preferably, the method is semi-quantitative or quantitative. Suitable methods are described in the following.

First, binding of a ligand may be measured directly, e.g. by NMR or surface plasmon resonance.

Second, if the ligand also serves as a substrate of an enzymatic activity of the peptide or polypeptide of interest, an enzymatic reaction product may be measured (e.g. the amount of a protease can be measured by measuring the amount of cleaved substrate, e.g. on a Western Blot). For measurement of enzymatic reaction products, preferably the amount of substrate is saturating. The substrate may also be labeled with, a detectable label prior to the reaction. Preferably, the sample is contacted with the substrate for an adequate period of time. An adequate period of time refers to the time necessary for an detectable, preferably measurable amount of product to be produced. Instead of measuring the amount of product, the time necessary for appearance of a given (e.g. detectable) amount of product can be measured.

Third, the ligand may be coupled covalently or non-covalently to a label allowing detection and measurement of the ligand. Labeling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. Said secondary ligand may be coupled with a suitable label and/or be the target (receptor) of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.)

The ligand or substrate may also be “tagged” with one or more tags as known in the art. Such tags may then be targets for higher order ligands. Suitable tags include biotin, digoxigenin, His-tag, glutathione-S-transferase, FLAG, GFP mye-tag, influenza A virus hemagglutinin (IIA), maltose binding protein, and the like. In the case of a peptide or polypeptide, the tag is preferably at the N-terminus and/or C-terminus.

Suitable labels are any labels detectable by an appropriate detection method. Typical labels include gold particles, latex beads, acridan ester, luminol, ruthenium, enzymatically active labels, radioactive labels, magnetic labels (“e.g. magnetic beads”, including paramagnetic and superparamagnetic labels), and fluorescent labels.

Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star™ (Amersham Biosciences), ECF™ (Amersham Biosciences). A suitable enzyme-substrate combination may result in a colored reaction product, fluorescence or chemiluminescence, which can be measured according to methods known in the art (e.g. using a light-sensitive film or a suitable camera system). As for measuring the enzymatic reaction, the criteria given above apply analogously.

Typical fluorescent labels include fluorescent proteins (such as GFP and its derivatives, Cy3, Cy5, Texas Red, Fluorescein, and the Alexa dyes (e.g. Alexa 568). Further florescent labels are available e.g. from Molecular Probes (Oregon). Also the use of quantum dots as fluorescent labels is contemplated.

Typical radioactive labels include 35S, 125I, 32P, 33P and the like. A radioactive label can be detected by any method known and appropriate, e.g. a light-sensitive film or a phosphor imager.

Suitable measurement methods according the present invention also include precipitation (particularly immunoprecipitation), electrochemiluminescence (electro-generated chemiluminescence), RIA radioimmunoassay), ELISA (enzyme-linked, immunosorbent assay), sandwich enzyme immune tests, electrochemiluminescence sandwich immunoassays (ECLIA), dissociation-enhanced lanthanide fluoro immunoassay (DELFIA), scintillation proximity assay (SPA), turbidimetry, nephelometry, latex-enhanced turbidimetry or nephelometry; solid phase immune tests, and mass spectrometry such as SELDI-TOF, MALDI-TOF, or capillary electrophoresis-mass spectrometry (CE-MS). Further methods known in the art (such as gel electrophoresis, 2D gel electrophoresis, SDS polyacrylamide gel electrophoresis (SDS-PAGE), Western Blotting), can be used alone or in combination with labeling or other detection, methods as described above.

Furthermore, suitable methods include microplate ELISA-based methods, fully-automated or robotic immunoassays (available for example on ELECSYS analyzers, Roche Diagnostics GmbH), CBA (an enzymatic cobalt binding assay, available for example on Roche/Hitachi analyzers), and latex agglutination assays (available for example on Roche Hitachi analyzers).

Preferred ligands include antibodies, nucleic acids, peptides or polypeptides, and aptamers, e.g. nucleic acid or peptide aptamers. Methods to such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g. phage display.

The term “antibody” as used herein includes both polyclonal and monoclonal antibodies, as well as fragments thereof, such as Fv, Fab and F(ab)₂ fragments that are capable of binding antigen or hapten.

In another preferred embodiment, the ligand, preferably chosen from the group consisting of nucleic acids, peptides, polypeptides, more preferably from the group consisting of nucleic acids, antibodies, or aptamers, is present on an array.

Said array contains at least one additional ligand, which may be directed against a peptide, polypeptide or a nucleic acid of interest. Said additional ligand may also be directed against a peptide, polypeptide a nucleic acid of no particular interest in the context of the present invention. Preferably, ligands for at least three, preferably at least five, more preferably at least eight peptides or polypeptides of interest in the context of the present invention are contained on the array.

Binding of the ligand on the array may be detected by any known readout or detection method, e.g. methods involving optical (e.g. fluorescent), electrochemical, or magnetic signals, or surface plasmon resonance.

According to the present invention, the term “array” refers to a solid-phase or gel-like carrier upon which at least two compounds are attached or bound in one-, two- or three-dimensional arrangement. Such arrays (including “gene chips”, “protein chips,”, antibody arrays and the like) are generally known to the person skilled in the art and typically generated on glass microscope slides, specially coated glass slides such as polycation-, nitrocellulose- or biotin-coated slides, cover slips, and membranes such as, for example, membranes based on nitrocellulose or nylon. The array may include a bound ligand or at least two cells expressing each at least one ligand.

It is also contemplated to use “suspension arrays” as arrays according to the present invention (Nolan J P, Sklar L A. (2002). Suspension array technology: evolution of the flat-array paradigm. Trends Biotechnol. 20(1):9-12). In such suspension arrays, the carrier, e.g. a microbead or microsphere, is present in suspension. The array consists of different microbeads or microspheres, possibly labeled, carrying different ligands.

The invention further relates to a method of producing arrays as defined above, wherein at least one ligand is bound to the carrier material in addition to other ligands.

Methods of producing such arrays, for example based on solid-phase chemistry and photo-labile protective groups, are generally known (U.S. Pat. No. 5,744,305). Such arrays can also be brought into contact with substances or substance libraries and tested for interaction, for example for binding or change of confirmation. Therefore, arrays comprising a peptide or polypeptide as defined above may be used for identifying ligands binding specifically to said peptides or polypeptides.

Peptides and polypeptides (proteins) can be measured in tissue, cell, and body fluid samples, i.e. preferably in vitro. Preferably, the peptide or polypeptide of interest is measured in a body fluid sample.

A tissue sample according to the present invention refers to any kind of tissue obtained from the dead or alive human or animal body. Tissue samples can be obtained by any method known to the person skilled in the art, for example by biopsy or curettage.

Body fluids according to the present invention may include blood, blood serum, blood plasma, lymphe, cerebral liquor, saliva, vitreous humor, and urine. Particularly, body fluids include blood, blood serum, blood plasma, and urine. Samples of body fluids can be obtained by any method known in the art.

Some of the samples, such as urine samples, may only contain degradation products, in particular fragments, of the peptide or polypeptide of interest. However, as laid out above, measurement of the level may still be possible as long as the fragments are specific for the peptide or polypeptide of interest.

If necessary, the samples may be further processed before measurement. For example, nucleic acids, peptides or polypeptides may be purified from the sample according to methods known in the art, including filtration, centrifugation, or extraction methods such as chloroform/phenol extraction.

Furthermore, it is contemplated to use so called point-of-care or lab-on-a-chip devices for obtaining the sample and measuring the peptide or polypeptide of interest. Such devices may be designed analogously to the devices used in blood glucose measurement. Thus, a patient will be able to obtain the sample and measure the peptide or polypeptide of interest without immediate assistance of a trained physician or nurse.

In another preferred embodiment, the present invention relates to a kit comprising (a) a means or device for measuring the level of an ANP-type peptide in a sample from a subject, and (b) a means or device for measuring the level of a BNP-type peptide in a sample from a subject. Preferably, the means according to (a) is a ligand binding specifically to the ANP-type peptide, and/or the means according to (b) is a ligand binding specifically to the BNP-type peptide. In another preferred embodiment, the present invention relates to the use of such a kit for diagnosing a cardiac dysfunction in a subject. In another preferred embodiment, the present invention relates to the use of such a kit for diagnosing the presence or severity of a diastolic dysfunction in a subject.

In another preferred embodiment, the present invention relates to the use of a ligand specifically binding NT-proANP) and/or a ligand specifically binding to NT-proBNP for the manufacture of a diagnostic kit for diagnosing a cardiac dysfunction, preferably a diastolic dysfunction. In another preferred embodiment, the diagnostic kit is for distinguishing a diastolic dysfunction from a systolic dysfunction.

Optionally, the kit may additionally comprise a user's manual for interpreting the results of any measurement(s) with respect to diagnosing a cardiac dysfunction, preferably a diastolic dysfunction. In another preferred embodiment, the user's manual is for interpreting the results of any measurements(s) with respect to distinguishing a diastolic dysfunction from a systolic dysfunction. Particularly, the user's manual may include information about what measured level corresponds to what kind of dysfunction. This is outlined in detail elsewhere in this specification. Additionally, such user's manual may provide instructions about correctly using the components of the kit for measuring the level(s) of the respective biomarkers.

In another preferred embodiment, the present invention relates to diagnosing the risk of a patient of suffering from a cardiac disease. According to the present invention, the term “risk” relates to the probability of a particular incident, more particularly a cardiovascular complication or heart failure, to take place. If a method according to the present invention indicates that the subject is suffering from a cardiac dysfunction, then it also indicates that the subject is at risk of suffering from a more severe cardiac dysfunction. For example if a method according to the present invention indicates that the subject is suffering from diastolic dysfunction, then the method also indicates that the subject is at risk of suffering from diastolic heart failure. In another example, if a method according to the present invention indicates that the subject is suffering from less severe diastolic dysfunction, then the method indicates that the subject is at risk of suffering a more severe diastolic dysfunction.

The present invention also relates to methods of treatment of cardiac dysfunctions or to methods for deciding about whether a subject requires treatment of a cardiac dysfunction. In general, if a method according to the present invention indicates the presence of a cardiac dysfunction or a risk of suffering from a cardiac dysfunction, then it is preferably decided that the subject requires treatment of the cardiac dysfunction.

If a method according to the present invention indicates that a cardiac dysfunction is present in the subject or that the subject is at risk of suffering from a cardiac dysfunction, then treatment may be initiated or adapted. The level(s) and/or ratio(s) of the ANP-type and BNP-type peptides in subject may be monitored at regular intervals. Furthermore, the subject may be investigated intensively by further diagnosis according to methods known to the skilled cardiologist, such as electrocardiography, or echocardiography. Treatment may include any measures which generally are associated with reducing the risk of suffering from cardiac dysfunction or heart failure. E.g., treatment with non-steroidal anti-inflammatory drugs (e.g. Cox-2 inhibitors or selective Cox-2 inhibitors such as celecoxib or rofecoxib) may be discontinued or the dosage of any such drugs administered may be reduced. Other possible measures are restriction of salt-intake regular moderate exercise, providing influenzal and pneumococcal immunization, surgical treatment (e.g. revascularization, balloon dilatation, stenting, by-pass surgery), administering drugs such as diuretics (including co-administration of more than one diuretic), ACE (angiotensin converting enzyme) inhibitors, B-adrenergic blockers, aldosterone antagonists, calcium antagonists (e.g. calcium channel blockers), angiotensin-receptor blockers digitalis, as well as any other measures known and deemed appropriate by the person skilled in the art.

More particularly, in a further embodiment, the present invention relates to a method for deciding on the possible treatment of a subject for a cardiac dysfunction, comprising (a) measuring, preferably in vitro, the level of an ANP-type peptide in a sample from the subject, (b) measuring, preferably in vitro, the level of a BNP-type peptide in a sample from the subject, (c) calculating the ratio of the measured level of the ANP-type peptide to the measured level of the BNP-type peptide, (d) comparing the calculated ratio to at least one known ratio indicative of the presence or absence of a cardiac dysfunction, (e) optionally initiating an examination of the patient by a cardiologist, (f) recommending the initiation of the treatment or refraining from the treatment, optionally in consideration of the result of the patient's examination by the cardiologist. Preferably, initiating an examination by a cardiologist and/or initiating treatment is recommended if the method indicates the presence of a cardiac dysfunction. The method relates to all dysfunctions mentioned earlier in this specification, particularly to initiating treatment of a diastolic dysfunction. It is evident that the method may be adapted according to all embodiments or preferred aspects of the invention mentioned in this specification.

### SPECIFIC EMBODIMENTS

### Example 1

### Measurement of NT-proBNP

NT-proBNP can be determined by an electrochemiluminescence immunoassay (ELECSYS proBNP sandwich immunoassay; Roche Diagnostics, Mannheim, Germany) on ELECSYS 2010. The assay works according to the electrochemiluminescence sandwich immunoassay principle. In a first step, the biotin-labeled IgG (1-21) capture antibody, the ruthenium-labeled F(ab′)2 (39-50) signal antibody and 20 microliters of sample are incubated at 37 C for 9 minutes. Afterwards, streptavidin-coated magnetic microparticles are added and the mixture is incubated for additional 9 minutes. After the second incubation, the reaction mixture is transferred to the measuring cell of the system where the beats are magnetically captured onto the surface of an electrode. Unbound label is removed by washing the measuring cell with buffer.

In the last step, voltage is applied to the electrode in the presence of a tri-propylamine containing buffer and the resulting electrochemiluminescent signal is recorded by a photomultiplier. All reagents and, samples are handled fully automatically by the ELECSYS instrument. Results are determined via a calibration curve which is instrument-specifically generated by 2-point calibration and a master curve provided via the reagent barcode. The test is performed according to the instructions of the manufacturer.

Blood for hormone analysis may be sampled in EDTA-tubes containing 5000 U aprotinine (Trasylol, Beyer, Germany) and Lithium-Heparin-tubes (for clinical chemistry), as appropriate. Blood and urine samples are immediately spun for 10 min. at 3400 rpm at 4 C. Supernatants are stored at −80° C. until analysis.

### Measurement of N-proANP

NT-proANP can be determined by a competitive-binding radioimmunoassay with magnetic solid phase technique in a modification of Sundsfjord, J. A., Thibault, G., et al. (1988). Identification and plasma concentrations of the N-terminal fragment of proatrial natriuretic factor in man. J Clin Endocrinol Metab 66:605-10, using the same rabbit-anti-rat proANP polyclonal serum, human proANP (1-30) from Peninsula Lab (Bachem Ltd, St. Helene, UK) as the standard, and iodined, pro-ANP 1-30 purified by HPLC for radio labeling. In order to achieve high sensitivity and good precision, Dynabeads M280 with sheep-anti-rabbit IgG (Dynal Biotech, Oslo, Norway) as solid phase and second antibody may be used.

### Example 2

A total of 542 (315 male, 227 female) elderly (more than 65 year-old) patients which had mild symptoms of breathing difficulties were included in a study related to the prognostic value of NT-proBNP). The median age was 63±11 years. In 454 patients of this group the levels of NT-proBNP and NT-proANP was measured. All patients received a clinical investigation, electrocardiogram, and an echocardiogram. Diastolic dysfunction was estimated by analyzing the ratio of E-wave to A-wave as described in (Aurigemma and Gaasch (2004), cited above). A systolic dysfunction was diagnosed-if an LVEF of less than 50% was measured. Patients without impaired systolic function were grouped according to the degree of the diastolic dysfunction as estimated according to the ratio of E-wave to A-wave (Aurigemma and Gaasch (2004), cited above).

TABLE 1
Plasma levels of natriuretic peptides in certain conditions

| | | | ratio of | |
|---|---|---|---|---|
| | | | NT-proANP | |
| | | | (pg/ml) to | |
| | NT-proBNP | NT-proANP | NT-proBNP | |
| Dysfunction | (pg/ml) | (pg/ml) | (pg/ml) | N |
| no DD | 122 ± 13 | 3270 ± 172 | 26.8 | 88 |
| less severe DD | 177 ± 11 | 3216 ± 98 | 18.17 | 307 |
| more severe DD | 437 ± 144 | 4130 ± 264 | 9.45 | 59 |
| systolic dysfunction | 1068 ± 619 | 4233 ± 541 | 3.96 | 16 |

DD, diastolic dysfunction;
N, number of subjects analyzed

It can be seen that NT-proBNP rises more steeply than NT-proANP with an increase of the cardiac dysfunction (from no DD, to less severe DD, to more severe DD, to systolic dysfunction). Furthermore, it can be seen that the ratio of NT-proANP and NT-proBNP can be used to diagnose character and extent of the cardiac dysfunction.

### Example 3

In a sequential study, the study subjects received the following examinations: (1) coronary angiography for diagnosing coronary heart disease, (2) echocardiography, particularly for assessing and estimating a systolic dysfunction, electrocardiogram for assessing the existence of previous infarction, arrhythmias, or any other information.

The patients were grouped according to the underlying disease, and the levels of NT-proBNP and NT-proANP were measured.
Group 1: All subjects with coronary heart disease as determined by angiography
Group 2: Valve defects of various kinds, e.g. mitral valve defects
Group 3: Dilatative cardiomyopathy
Group 4: Hypertrophic cardiomyopathy
Group 5: Subjects without coronary heart disease (healthy)
Group 6: Patients not belonging to any of the other groups, e.g. having arrhythmias.

Further analysis was performed relating to present or absent systolic dysfunction, age, atrial function, and arrhythmias, e.g. atrial arrhythmia.

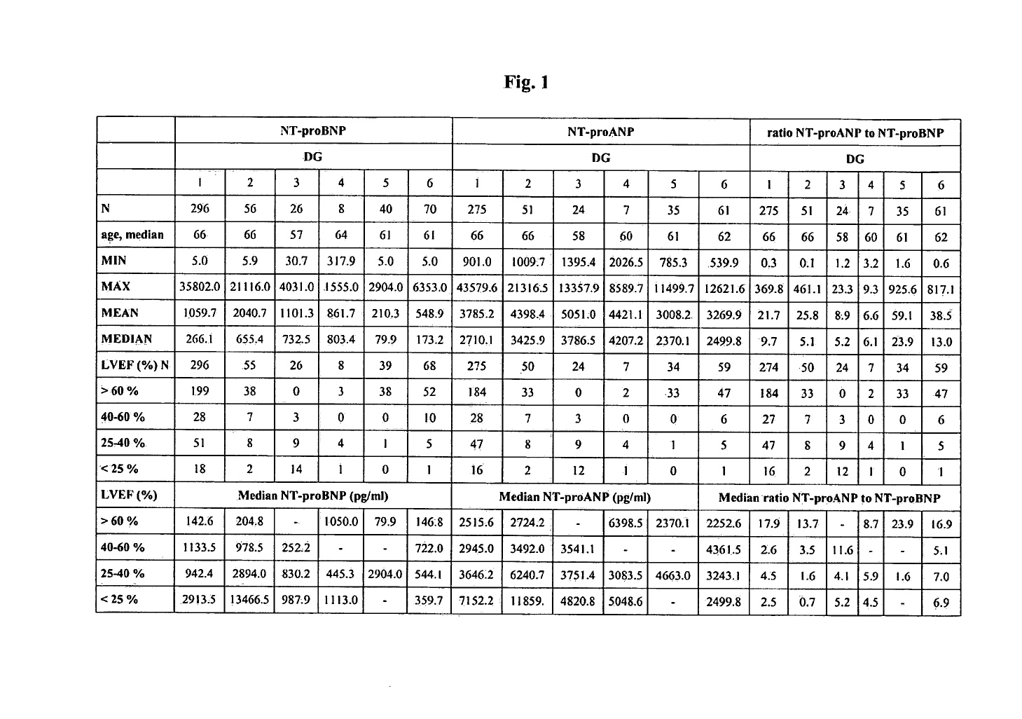

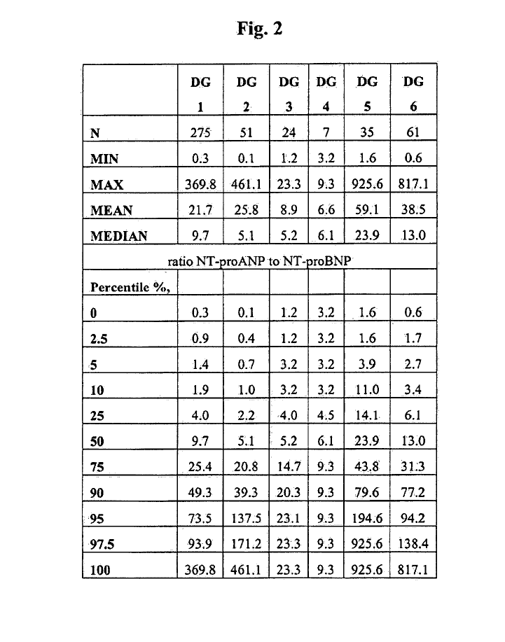

As can be seen from FIGS. 1 and 2, the ratio of NT proANP to NT-proBNP levels is dependent on the LVEF in all groups. In the group of valve defects (group 2) the NT-pro ANP levels tend to be higher. Groups 3 and 4 are somewhat unusual groups.

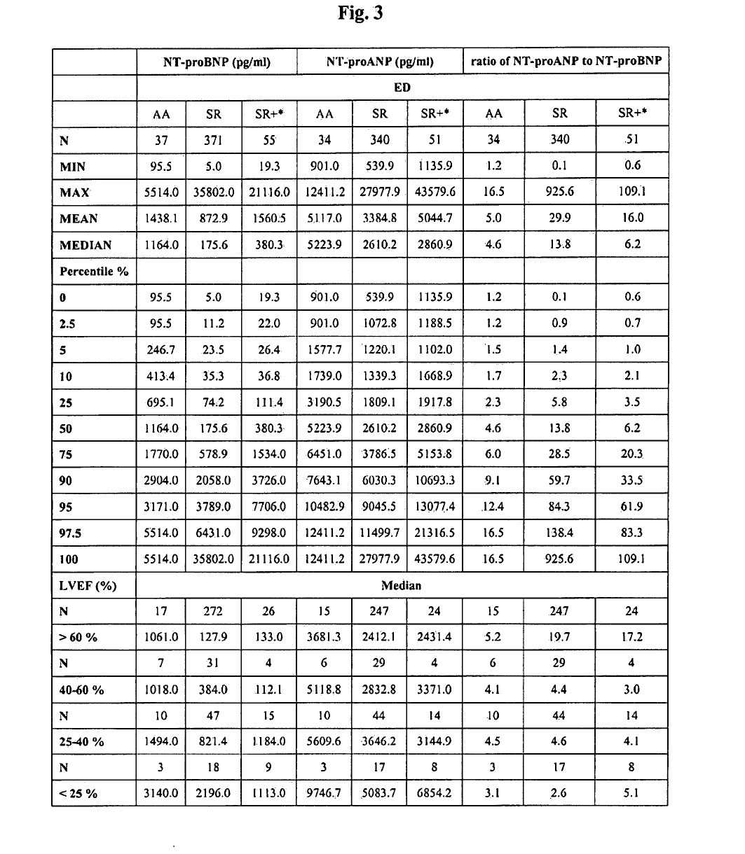

In a further analysis (see FIG. 3), the underlying disease was not taken into account and simply those patients were analyzed which had atrial, arrhythmia and can be recognized as fibrillation arrhythmia (AA). Patients with sinus rhythm are generally healthier. Sinus rhythm is depicted as “SR”. Patients with sinus rhythm and simultaneous further electrocardiogram abnormalities (e.g. right bundle branch block, left bundle branch block, or similar disorders) are depicted as “SR+”.

In patients with atrial fibrillation (fibrillation arrhythmia), a lower ratio of NT-proANP to NT-proBNP was found than in the group with sinus rhythm.

### Example 4

Patients suspected of having coronary heart disease were subjected to physical strain or artificial cardiac strain evoked by medicaments. In patients with coronary heart disease, the strain will result in pain and/or changes in the electrocardiogram. In the present study, the patients were also analyzed by thallium scintigraphy. The thallium scintigram allows to recognize whether strain causes ischemia. The results were grouped as ischemia not being detectable, being persistent, or being reversible. A shown in Table 2, subjects without ischemia had significantly lower NT-proBNP and NT-proANP levels.

TABLE 2

| | | | |
|---|---|---|---|
| | | | ischemia |
| no signs of ischemia | ischemia (total) | ischemia (persistent) | (reversible) |
| (N = 61) | (N = 78) | (N = 54) | (N = 24) |

Median NT-proANP, pg/ml

| | | | |
|---|---|---|---|
| 2566.392 | 4750.63 | 4610.39 | 5153.82 |

Median NT-proBNP, pg/ml

| | | | |
|---|---|---|---|
| 139 | 484 | 535 | 327 |

ratio of NT-proANP to NT-proBNP

| | | | |
|---|---|---|---|
| 18.5 | 9.8 | 8.6 | 15.7 |

Furthermore, the ratio of the levels of NT-proANP to NT-proBNP were significantly higher in patients without ischemia than in patients with reversible ischemia.

Patients showing ischemia have a coronary heart disease which is expressed predominantly in an impairment of cardiac function due to an earlier cardiac damage. Therefore, in these patients the presence of a diastolic or systolic dysfunction can be assumed, which is also expressed in the low ratio of NT-proANP to NT-proBNP.

Patients showing no ischemia in the thallium scintigram have no significant arteriosclerosis and consequently usually no significantly impaired cardiac function.

What is claimed is:

1. A method for diagnosing the presence or absence of a diastolic dysfunction in human subject comprising the steps of
measuring in vitro a level of N-terminal pro atrial natriuretic peptide (NT-proANP) in a sample from the subject,
measuring in vitro a level of N-terminal pro brain natriuretic peptide (NT-proBNP) in a sample from the subject,
calculating a ratio of the level of the NT-proANP to the level of the NT-proBNP, and
comparing the calculated ratio to at least one known ratio of NT-proANP to NT-proBNP as a measure of the presence or absence of the diastolic dysfunction.

2. The method of claim 1 wherein the sample is plasma.

3. The method of claim 1 wherein the at least one known ratio is 6 to 20 (pg/ml of NT-proANP to pg/ml of NT-proBNP), which is indicative of the presence of a diastolic dysfunction.

4. A method for diagnosing a severity of a diastolic dysfunction in a human subject comprising the steps of
measuring in vitro a level of N-terminal pro atrial natriuretic peptide (NT-proANP) in a sample from the subject,
measuring in vitro a level of N-terminal pro brain natriuretic peptide (NT-proBNP) in a sample from the subject,
calculating a ratio of the level of the NT proANP to the level of the NT-proBNP, and
comparing the calculated ratio to at least one known ratio of the ANP peptide to the BNP peptide as a measure of the severity of the diastolic dysfunction.

5. The method of claim 4 wherein the sample is plasma.

6. The method of claim 4 wherein the at least one known ratio correlates inversely with the severity of the diastolic dysfunction and a ratio in the range of 15 to 20 (pg/ml of NT-proBNP to pg/ml of) NT-proBNP) indicates a loss severe diastolic dysfunction and a ratio in the range of 6 to 15 (pg/ml of NT-proANP to pg/ml of NT-proBNP) indicates a more severe diastolic dysfunction.

7. A method for diagnosing the presence of a systolic dysfunction in a human subject comprising the steps of
measuring in vitro a level of N-terminal pro atrial natriuretic peptide (NT-proANP) in a sample from the subject,
measuring in vitro a level of N-terminal pro brain natriuretic peptide (NT-proBNP) in a sample from the subject,
calculating a ratio of the level of the NT-proANP to the level of the NT-proBNP, and
comparing the calculated ratio to at least one known ratio of the ANP peptide to the BNP peptide as a measure of diagnosing the presence of a systolic dysfunction.

8. The method of claim 7 wherein the sample is plasma.

9. The method of claim 7 wherein the at least one known ratio of NT-proBNP is less than 4.5, which indicates the presence of a systolic dysfunction.

10. A method for diagnosing a risk of diastolic heart failure in a human subject comprising the steps of
measuring in vitro a level of N-terminal pro atrial natriuretic peptide (NT-proANP) in a sample from the subject,
measuring in vitro a level of N-terminal pro brain natriuretic peptide (NT-proBNP) in a sample from the subject,
calculating a ratio of the level of the NT-proANP to the level of the NT-proBNP, and
comparing the calculated ratio to at least one known ratio of the ANP peptide to the BNP peptide as a measure of the risk of diastolic heart failure.

11. The method of claim 10 wherein the sample is plasma.

12. A kit for diagnosing the presence or absence or severity of a diastolic dysfunction in a human subject comprising
a means or device for measuring a level of N-terminal pro atrial natriuretic peptide (NT-proANP) in a sample from a subject,
a means or device for measuring a level of N-terminal pro brain natriuretic peptide (NT-proBNP) in a sample from a subject, and
instructions for performing the measurements, calculating a ratio of the measured level of NT-proANP to the measured level of NT-proBNP, and interpreting the calculated ratio with respect to diagnosing the presence or absence or severity of the diastolic dysfunction.

13. The kit of claim 12 wherein the means for measuring a level of NT-proANP comprises a ligand binding specifically to NT-proANP and wherein the means for measuring a level of NT-proBNP comprises a ligand binding specifically to NT-proBNP.
